(19) United States
(12) Patent Application Publication
(10) Pub. No.: US 2024/0274262 A1
(43) Pub. Date: Aug. 15, 2024

(54) OUTPUT APPARATUS AND RADIOTHERAPY APPARATUS

(71) Applicant: Canon Medical Systems Corporation, Otawara-shi (JP)

(72) Inventor: Shinji HASEGAWA, Nasushiobara (JP)

(73) Assignee: Canon Medical Systems Corporation, Otawara-shi (JP)

(21) Appl. No.: 18/437,064

(22) Filed: Feb. 8, 2024

(30) Foreign Application Priority Data
Feb. 9, 2023 (JP) 2023-018666

(51) Int. Cl. G16H 20/40; A61N 5/10

(52) U.S. Cl. CPC G16H 20/40; A61N 5/1081

(57) ABSTRACT

An output apparatus includes processing circuitry. The processing circuitry is configured to: acquire identification information for identifying a state of a patient fastened to a radiotherapy apparatus with a fastener; calculate a rest level of the patient based on the identification information; and output a change in the rest level over time.

### CROSS-REFERENCE TO RELATED APPLICATIONS

This application is based upon and claims the benefit of priority from Japanese Patent Application No. 2023-018666, filed Feb. 9, 2023, the entire contents of which are incorporated herein by reference.

### FIELD

Embodiments described herein relate generally to an output apparatus and a radiotherapy apparatus.

### BACKGROUND

In radiotherapy using a radiotherapy apparatus, there is a case where a treatment on a patient is performed while the patient is immobilized by fastening the patient with a fastener. In such a case, the patient may feel discomfort by being deprived of freedom by the fastener. The patient feeling discomfort may feel anxious and/or sick. There is also a case where it is difficult for the patient to express his or her intention regarding such a situation. In particular, if the patient is a child, he or she may not be able to express his or her intention regarding anxiety and/or sickness. In such a case, there is a possibility that the operator of the radiotherapy apparatus may perform a treatment on the patient feeling discomfort, without noticing the anxiety and/or sickness of the patient.

### BRIEF DESCRIPTION OF THE DRAWINGS

FIG. 1 is a diagram showing an example of a configuration of a radiotherapy apparatus according to a first embodiment.

FIG. 2 is a diagram showing a state of a treatment room in which the radiotherapy apparatus according to the first embodiment is installed.

FIG. 3 is a flowchart illustrating a procedure for a treatment support process by the radiotherapy apparatus according to the first embodiment.

FIG. 4 is a diagram showing an example of a display screen displayed by the radiotherapy apparatus according to the first embodiment.

FIG. 5 is a diagram showing an example of a configuration of a radiotherapy apparatus according to a second embodiment.

FIG. 6 is a flowchart illustrating a procedure for a treatment support process by the radiotherapy apparatus according to the second embodiment.

FIG. 7 is a diagram showing an example of a configuration of a radiotherapy apparatus according to a third embodiment.

FIG. 8 is a flowchart illustrating a procedure for a treatment support process by the radiotherapy apparatus according to the third embodiment.

FIG. 9 is a diagram showing an example of a display screen displayed by the radiotherapy apparatus according to the third embodiment.

### DETAILED DESCRIPTION

In general, according to one embodiment, an output apparatus includes processing circuitry. The processing circuitry is configured to: acquire identification information for identifying a state of a patient fastened to a radiotherapy apparatus with a fastener; calculate a rest level of the patient based on the identification information; and output a change in the rest level over time.

Hereinafter, an output apparatus and a radiotherapy apparatus according to an embodiment will be described in detail with reference to the accompanying drawings. In the following description, constituent elements having substantially the same functions and configuration will be denoted by the same reference numerals, and a repeat description will be made only when necessary.

### First Embodiment

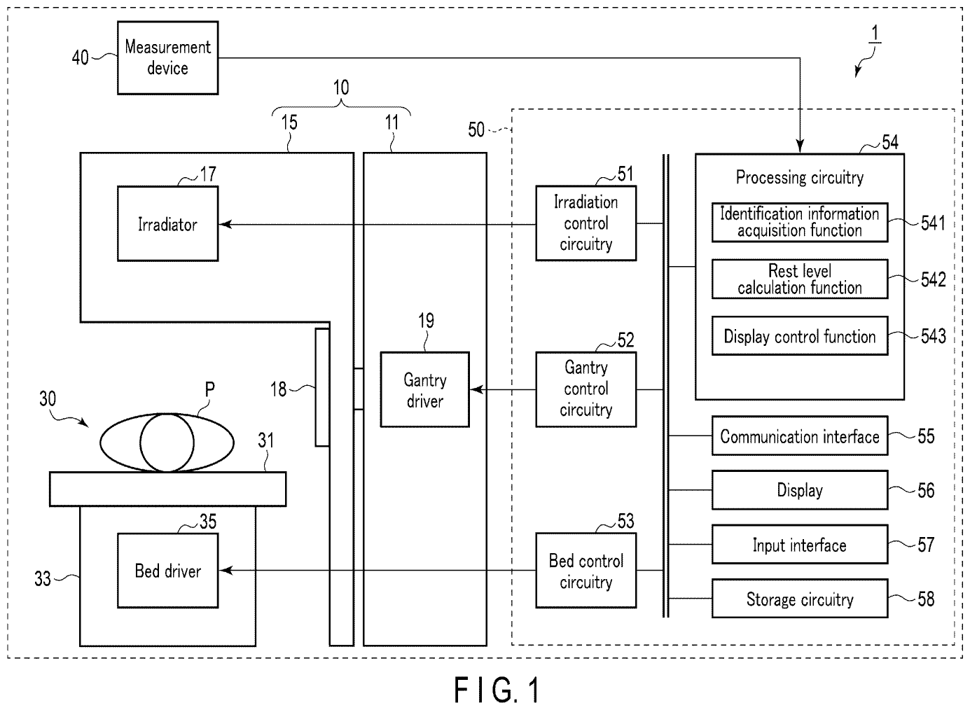

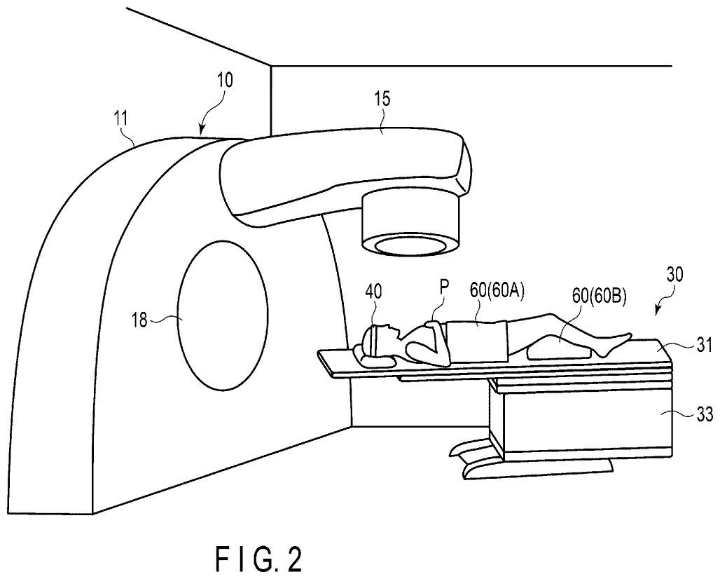

FIG. 1 is a diagram showing a configuration of a radiotherapy apparatus 1 including an output apparatus according to the present embodiment. FIG. 2 is a diagram showing a state of a treatment room in which the radiotherapy apparatus 1 is installed. The radiotherapy apparatus 1 according to the present embodiment is configured to calculate a rest level indicating an index of a sense of discomfort being felt by a patient and to display a change in the rest level over time, in order to allow an operator to grasp the situation of the patient at the time of setting up the patient using a fastener, even if his or her intention is not expressed. The rest level is a numerical index indicating a sense of discomfort being felt by a patient who is receiving radiotherapy. With the operator checking the rest level of the patient in real time at the time of fastening of the patient with a fastener, it is possible to inhibit a treatment from being started on a patient feeling discomfort. Hereinafter, a case will be described where the rest level is set in such a manner that the smaller the sense of discomfort being felt by the patient, namely, the more at rest the patient is, the larger the value of the rest level becomes. However, the rest level may be set in such a manner that the greater the sense of discomfort being felt by the patient, namely, the less at rest the patient is, the larger the value of the rest level becomes.

As shown in FIGS. 1 and 2, the radiotherapy apparatus 1 includes a gantry 10, a bed 30, a console 50, and a measurement device 40. The gantry 10 and the bed 30 are installed in a treatment room. The gantry 10 is installed on, for example, a wall surface of the treatment room. The console 50 is installed in, for example, an operation room adjacent to the treatment room.

The gantry 10 includes a gantry main body 11, an irradiation head 15, and a display 18. The gantry main body 11 rotatably supports the irradiation head 15. An irradiator 17 is incorporated in the irradiation head 15. The irradiator 17 includes an acceleration tube (not illustrated) for accelerating electrons, etc. generated by an electron gun, etc., and a metal target (not illustrated) with which electrons accelerated by the acceleration tube collide. Through collision of electrons with the metal target, X-rays are generated as radiation. Upon receiving a control signal supplied from the console 50, the irradiator 17 emits radiation. The irradiator 17 is provided with a multi-leaf collimator (MLC; not illustrated) at its radiation emission port. The multi-leaf collimator supports a plurality of leaves formed of an X-ray shielding material such that the leaves can be moved individually. By moving the leaves, an irradiation field can be formed in a desired shape. The irradiator 17 is an example of an irradiation unit.

The radiation that can be emitted by the irradiator 17 is not limited to X-rays. The irradiator 17 may emit any radiation such as electron rays and particle rays.

The bed 30 includes a base 33 and a top plate 31. The base 33 movably supports the top plate 31. A patient P is placed on the top plate 31. For example, the base 33 movably supports the top plate 31 along its longitudinal axis.

At the time of radiotherapy, the patient P is fastened onto the top plate 31 with a fastener 60. The fastener 60 is installed between, for example, the patient P and the top plate 31. The fastener 60 is designed to fasten a site to be treated, such as a head, an abdomen, a leg, etc., of the patient P. The fastener 60 is designed, for example, to cover the body of the patient P from above, thereby preventing the movement of the patient. The fastener 60 may be shaped according to the physical structure of the patient. Moreover, if the fastener 60 is for a head, it may be formed of a mesh-like member. Furthermore, the fastener 60 may be used either alone, or two or more fasteners 60 may be used in combination. The fastener 60 may be called a restraint, a fixing tool, a fixture, or an immobilization device.

The measurement device 40 measures information (hereinafter referred to as “identification information”) for identifying the state of the patient P placed on the bed 30. The identification information is a plurality of indexes used to grasp the rest level of the patient P. The identification information includes, for example, vital signs, an oxygen saturation (SpO₂), beta wave observation results, an expression, a complexion, etc. The vital signs include, for example, a “respiratory rate”, a “body temperature”, a “blood pressure”, a “pulse (heart rate)”, a “consciousness level”, and a “urine output”. The measurement device 40 for measuring the vital signs is realized by, for example, general equipment such as respiratory waveform measurement equipment, blood pressure measurement equipment, electrocardiographic wave measurement equipment, and the like. Also, the measurement device 40 is realized by a device such as an electroencephalograph for observing beta waves or an optical camera for generating an image for determining the patient's expression and/or complexion. The identification information measured by the measurement device 40 is transmitted to the console 50. Also, a wearable terminal including a sensor to be attached to an arm or a forehead may be used. In this case, the effect on the patient caused by the measurement of the identification information can be suppressed to be low.

The gantry 10 is provided with a display 18. The display 18 is, for example, supported by the gantry main body 11, and is fixed so as not to rotate together with the irradiation head 15. The display 18 displays various types of information. The display 18 displays, for example, results of measurement by the measurement device 40, a change in the rest level over time calculated by the console 50, a procedure for supporting execution of radiotherapy, and the like. The display 18 is, for example, a liquid crystal display, a cathode ray tube (CRT) display, or the like. The display 18 is an example of a display unit.

The console 50 includes irradiation control circuitry 51, gantry control circuitry 52, bed control circuitry 53, processing circuitry 54, a communication interface 55, a display 56, an input interface 57, and storage circuitry 58. In the present embodiment, the console 50 including the processing circuitry 54 and the storage circuitry 58 corresponds to the output apparatus.

The irradiation control circuitry 51 controls the irradiator 17 in order to emit radiation in accordance with irradiation conditions included in a treatment plan. The irradiation conditions include an irradiation dose, an irradiation angle, etc. The irradiation conditions may be input by a user via the input interface 57. Data relating to the treatment plan is supplied from a treatment planning device (not illustrated), a radiotherapy information management system known as Oncology Information System (OIS), or the like. The irradiation control circuitry 51 includes a processor such as a central processing unit (CPU) and a memory such as a read-only memory (ROM) and a random access memory (RAM) as hardware resources.

The gantry control circuitry 52 controls the gantry driver 19 to allow the irradiation head 15 to be positioned at the irradiation angle included in the irradiation conditions. The gantry driver 19 is incorporated in, for example, the gantry main body 11 of the gantry 10. Upon receiving a control signal supplied from the gantry control circuitry 52, the gantry driver 19 rotates the irradiation head 15 around a rotation axis, and allows the irradiation head 15 to be positioned at the irradiation angle. The irradiation angle may be input by a user via the input interface 57. The gantry control circuitry 52 includes a processor such as a CPU and a memory such as a ROM, a RAM, etc., as hardware resources.

The bed control circuitry 53 controls the bed driver 35 to move the top plate 31 to a desired position. The bed driver 35 is incorporated in, for example, the base 33 of the bed 30. Upon receiving a control signal supplied from the bed control circuitry 53, the bed driver 35 moves the top plate 31. The bed control circuitry 53 includes a processor such as a CPU and a memory such as a ROM and a RAM as hardware resources.

The communication interface 55 performs data communications with the treatment planning device, the radiotherapy information management system, etc. via an unillustrated wired or wireless connection.

The display 56 displays various types of information. The display 56 outputs, for example, a graphical user interface (GUI) for receiving medical information generated by the processing circuitry 54 and various operations made by the operator. The display 56 is, for example, a liquid crystal display, a cathode ray tube (CRT) display, or the like. The display 56 is an example of a display unit.

The input interface 57 receives various input operations made by the operator, converts the received input operations into electric signals, and outputs them to the processing circuitry 54. The input interface 57 receives, for example, an input of medical information, inputs of various command signals, etc. from the operator. The input interface 57 is implemented by, for example, a mouse, a keyboard, a trackball and/or a switch button for performing various processes of the processing circuitry 54, a touch screen in which a display screen and a touchpad are integrated, non-contact input circuitry using an optical sensor, audio input circuitry, or the like. The input interface 57, which is connected to the processing circuitry 54, converts an input operation received from the operator into an electric signal, and outputs it to the control circuitry. Herein, the input interface does not necessarily include a physical operational component such as a mouse, a keyboard, etc. Examples of the input interface include electric signal processing circuitry that receives an electric signal corresponding to an input operation from an external input device provided separately from the apparatus, and outputs the electric signal to the processing circuitry 54. The input interface 57 is an example of an input unit.

The storage circuitry 58 is a storage device configured to store various types of information, such as a hard disk drive (HDD), a solid-state drive (SSD), or an integrated circuitry storage device. The storage circuitry 58 stores, for example, programs to be executed by the processing circuitry 54, a treatment plan supplied from the treatment planning device or the radiotherapy information management system, and the like. Also, the storage circuitry 58 may be a portable storage medium such as a compact disc (CD) or a digital versatile disc (DVD), as well as an HDD, an SSD, etc. The storage circuitry 58 may be a drive device configured to read and write various types of information from and to a semiconductor memory device such as a flash memory, a random access memory (RAM), etc. Also, the storage area of the storage circuitry 58 may be provided either in the radiotherapy apparatus 1, or in an external storage device connected via a network. The storage circuitry 58 may be referred to as a storage unit or a memory.

The storage circuitry 58 stores programs to be executed by the processing circuitry 54, various types of data to be used in processing by the processing circuitry 54, and the like. Examples of the programs that are used include programs installed on a computer from a network or a non-transitory computer readable storage medium, and configured to cause the computer to realize the functions of the processing circuitry 54. The various types of data to be described herein are typically digital data.

The processing circuitry 54 functions as the central nerve of the radiotherapy apparatus 1 and controls the overall operation of the radiotherapy apparatus 1. The processing circuitry 54 is a processor that executes programs stored in the storage circuitry 58, etc., and controls the components in accordance with the programs, thereby executing radiotherapy. Specifically, the processing circuitry 54 calls programs in the storage circuitry 58 to execute an identification information acquisition function 541, a rest level calculation function 542, and a display control function 543.

The processing circuitry 54 acquires, through the identification information acquisition function 541, identification information of the patient over time. For example, the processing circuitry 54 acquires identification information measured by the measurement device 40 every second or every minute. The processing circuitry 54, which implements the identification information acquisition function 541, is an example of an identification information acquisition unit.

The processing circuitry 54 calculates, through the rest level calculation function 542, a rest level of the patient over time using the identification information. The processing circuitry 54 calculates, for example, a rest level every time new identification information is acquired by the identification information acquisition function 541. The rest level is calculated by, for example, adding values weighted with different coefficients to parameters contained in the identification information. A formula for calculating the rest level is stored in, for example, the storage circuitry 58. Also, the rest level may be calculated by combining measurement values of the parameters, a degree of the change in the measurement value, an evaluation value obtained by converting analysis results into a numerical value, etc. For the calculation of the rest level, all the parameters contained in the identification information acquired through the identification information acquisition function 541 may be used, or only some of the parameters contained in the acquired identification information may be used. Also, the formula for calculating the rest level may be adjusted based on the operator's experience level, the operator's desire, and/or statistical information. The processing circuitry 54, which implements the rest level calculation function 542, is an example of a rest level calculation unit.

Also, the rest level may be calculated using a trained machine learning model. The trained machine learning model is realized by, for example, a neural network disclosed in Christopher M. Bishop, “Pattern Recognition and Machine Learning”, First Edition, Springer, U.S., 2006, pp. 225-290. The trained machine learning model may be any parameterized composite function that satisfies the above request. The trained machine learning model is a multilayer network model in which parameters are trained such that corresponding output data can be output based on input data. The multilayer network model is, for example, a deep neural network (DNN) that mimics a neural circuit of a biological brain. The multilayer network model is not limited to the DNN, and a convolutional neural network (CNN), for example, may be used as the multilayer network model.

If the rest level is calculated using a trained machine learning model, a machine learning model that has been trained to output a value of the rest level in response to an input of identification information is used. Examples of the identification information that is input to such a machine learning model include a measurement result, a frequency, and a stability of the “respiratory rate”, a measurement result and a variation of the “body temperature”, a measurement result and a variation of the “blood pressure”, a measurement result, a frequency, and a stability of the “pulse (heart rate)”, a determination result and a variation of the “consciousness level”, a value and a stability of an oxygen saturation (SpO₂), a photographed image of the face of a patient and an analysis result of an expression based on the photographed image. Examples of the training data used for training of such a machine learning model include the value of the rest level decided by a doctor or staff. A machine learning model trained to output a determination result regarding a level of discomfort of the patient using the rest level may be used, in addition to the value of the rest level. In this case, for the training data used for training of the machine learning model, a determination result regarding the level of discomfort of the patient determined by the doctor or staff is used, in addition to the value of the rest level decided by the doctor or staff.

With the display control function 543, the processing circuitry 54 causes the display 56 and/or the display 18 to display various types of information. For example, the processing circuitry 54 causes the display 56 and/or the display 18 to display the parameters of the identification information acquired by the measurement device 40 and a change in the value of the rest level over time calculated by the rest level calculation function 542. The processing circuitry 54 that implements the display control function 543 is an example of an output unit. The processing circuitry 54 that implements the display control function 543 may be referred to as a “display control unit”.

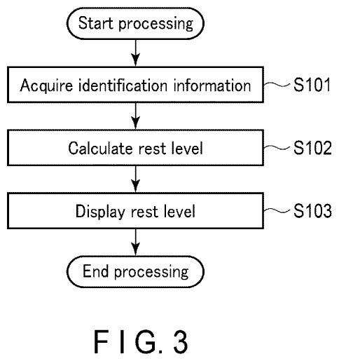

Next, an operation of a treatment support process executed by the radiotherapy apparatus 1 according to the present embodiment will be described. The treatment support process is a process of calculating and displaying a rest level of a patient in real time at the time of fastening the patient to the bed 30. The treatment support process is executed in a state in which the patient who has entered the treatment room is placed on the bed 30 and a measurement device 40 such as a wearable terminal is attached to the patient. FIG. 3 is a flowchart showing an example of a procedure of the treatment support process. The procedure in each process to be described below is given merely as an example, and each process may be suitably altered wherever possible. In the procedure to be described below, one or more steps may be suitably omitted, replaced, and/or added according to the embodiment.

### Treatment Support Process

### Step S101

The processing circuitry 54 acquires, through the identification information acquisition function 541, identification information for identifying the state of the patient P over time from the measurement device 40. The processing circuitry 54 acquires, from the measurement device 40, vital signs, an oxygen saturation, beta waves, a photographed facial image, etc. of the patient P. The acquired identification information is stored in the storage circuitry 58.

### Step S102

Next, the processing circuitry 54 calculates, through the rest level calculation function 542, a rest level over time of the patient P using the identification information acquired in the processing at step S101. The processing circuitry 54 calculates the rest level every few seconds, for example. At this time, the rest level may be calculated by reading the formula for calculating the rest level stored in the storage circuitry 58, or the rest level may be calculated by inputting identification information to the trained machine learning model stored in the storage circuitry 58.

### Step S103

Next, with the display control function 543, the processing circuitry 54 causes the display 18 to display the rest level calculated in the processing at step S102. At this time, the rest level is displayed on the display 18 in such a manner that its change over time can be identified.

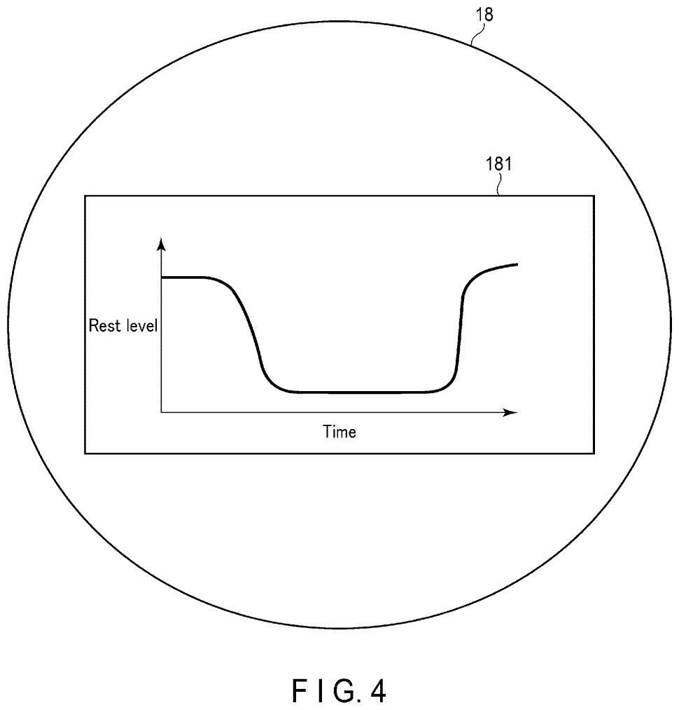

FIG. 4 is an example of a display screen displayed on the display 18. As shown in FIG. 4, a rest level display section 181 showing a change in the rest level over time, for example, is provided on the display screen. On the rest level display section 181, a graph showing a change in the rest level over time is displayed. In the graph showing the change in the rest level over time, the horizontal axis represents time and the vertical axis represents the rest level. The operator can quantitatively grasp the sense of discomfort being felt by the patient P by checking the change in the rest level over time being displayed on the display 18.

If, for example, the rest level has significantly decreased, as shown in FIG. 4, the operator can grasp that the sense of discomfort being felt by the patient P has increased, and can get rid of the cause of the sense of discomfort. If, for example, the fastener 60 had been attached immediately before a decrease of the rest level, it is determined that the fastener 60 has not been correctly attached, and the fastener 60 can be reattached. If the rest level increases after the reattaching of the fastener 60, as shown in FIG. 4, the operator can confirm that the sense of discomfort being felt by the patient P has decreased, and determine that the fastener 60 is the cause of the sense of discomfort. On the other hand, if the decreased rest level remains unchanged even after the fastener 60 is attached again, the operator grasps that fastener 60 is not the cause of the sense of discomfort, and takes measures against other causes.

Hereinafter, effects of the radiotherapy apparatus 1 including the output apparatus according to the present embodiment will be described.

The radiotherapy apparatus 1 including the output apparatus according to the present embodiment is capable of acquiring identification information for identifying a state of a patient fastened to the radiotherapy apparatus 1 with a fastener 60, calculating a rest level of the patient based on the identification information, and outputting a change in the rest level over time. For example, the change in the rest level over time can be output to the display 18 provided on the gantry 10 installed in a treatment room, to allow the display 18 to display the change in the rest level over time.

With the above configuration of the radiotherapy apparatus 1 according to the present embodiment, it is possible for the operator to grasp, even if a complaint has not been made by the patient who is feeling pain due to his or her skin being compressed or rubbed by the fastener 60, for example, that the patient is feeling discomfort by checking the change in the rest level over time being displayed on the display 18, and thereby to take quick and appropriate measures. Thus, since the operator can observe the state of the patient prior to emission of radiation in radiotherapy even if the patient's intention is not expressed, it is possible to set up the patient quickly and correctly, and to treat the patient in a comfortable state, while preventing a treatment to be started on a patient feeling discomfort.

An example has been described in which a change in the rest level is displayed in a graph; however, a change in the numerical value of the rest level may be displayed in a table format. Also, the numerical value or the graph of the rest level that is displayed may be color-coded according to the numerical value of the rest level.

In addition, a change in the rest level over time may be displayed on the display 56 provided on the console 50, instead of the display 18 provided on the gantry 10, and a change in the rest level over time may be displayed on both the display 18 and the display 56. Moreover, a change in the rest level over time may be output and displayed on another display device provided in the treatment room or operation room, and a change in the rest level over time may be output and displayed on a display device provided in another room in the medical facility.

Furthermore, the change in the rest level over time may be displayed on the display 18 during execution of radiotherapy (during emission of radiation), instead of during fastening of the patient with the fastener 60 prior to start of radiotherapy. In this case, the operator can grasp that the patient is feeling discomfort during a treatment, and can get rid of the cause of the sense of discomfort, thereby inhibiting the treatment to be executed on a patient feeling discomfort.

In addition, a change over time in the amount of change in the rest level relative to a reference value may be displayed in place of the change in the rest level over time. For the reference level, a rest level in a normal state, for example, is used. In this case, with the identification information acquisition function 541, the processing circuitry 54 acquires a rest level in a normal state as a reference value, calculates, with the rest level calculation function 542, a change amount in the current rest level relative to the reference value, and causes, with the display control function 543, the display 18 to display the calculated change amount. The “normal state” refers to a state in which the fastener 60 is not attached and the sense of discomfort being felt by the patient is small. The “normal state” indicates, for example, a state in which the patient is lying on the bed 30 during preparation of the fastener 60, a state (waiting state) in which the patient is waiting at a waiting room prior to radiotherapy, or a state immediately before the patient is placed on the bed 30 and the fastener 60 is attached. The reference value is calculated using identification information measured in the normal state, and is stored in advance in the storage circuitry 58. Moreover, the reference value may be calculated using identification information obtained from a patient to be treated, or using statistical values, etc. of reference values of other patients that have been calculated in the past. Furthermore, statistical values of reference values of patients of the same sex or race as the patient to be treated may be used.

By using a change amount in the rest level from the normal state, the operator can easily grasp how much the sense of discomfort being felt by the patient has changed due to attaching of the fastener 60. In addition, the reference value may be displayed on the display 18 together with the change in the rest level over time.

### Second Embodiment

A second embodiment will be described. In the present embodiment, the configurations of the first embodiment are modified in the following manner. A description of configurations, operations, and effects similar to those of the first embodiment will be omitted. In the present embodiment, a warning (an alert) is issued if the rest level has changed.

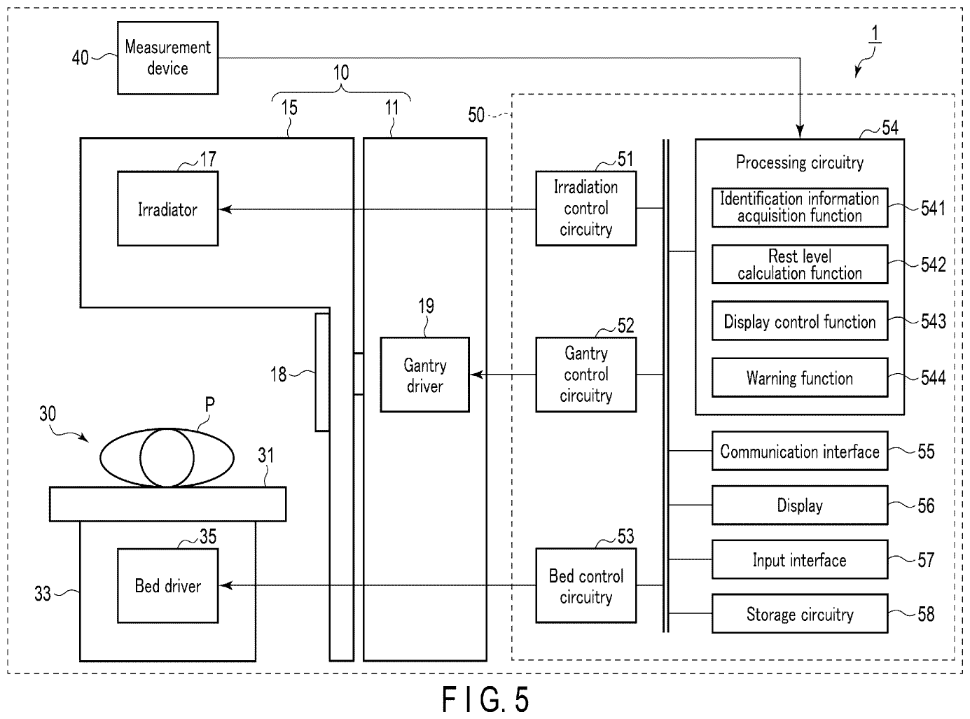

FIG. 5 is a diagram showing a configuration of a radiotherapy apparatus 1 according to the present embodiment. As shown in FIG. 5, in the present embodiment, the processing circuitry 54 further executes a warning function 544, in addition to the identification information acquisition function 541, the rest level calculation function 542, and the display control function 543. The processing circuitry 54, which implements the warning function 544, is an example of an output unit. The processing circuitry 54, which implements the warning function 544, may be referred to as a “warning unit”.

With the warning function 544, the processing circuitry 54 outputs a warning if the rest level has changed. Herein, a case will be described, as an example, where the rest level is set in such a manner that the smaller the sense of discomfort being felt by the patient, namely, the more the environment allows the patient to be at rest, the larger the value of the rest level becomes. With the warning function 544, the processing circuitry 54 warns the operator if the rest level has decreased below a predetermined threshold value. The threshold value is stored in advance in, for example, the storage circuitry 58. The threshold value may be altered by an input of the operator. Examples of the method of outputting a warning include a method of issuing a warning sound, a method of displaying a warning message on the display 18, and a method of reading a warning message with audio. As the warning, the color of the graph displaying the change in the rest level over time may be altered if the rest level has decreased below a threshold value.

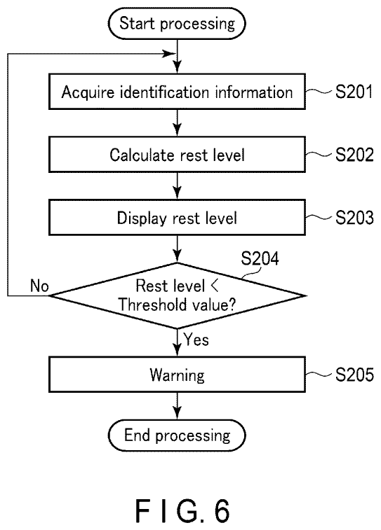

Next, an operation of a treatment support process executed by the radiotherapy apparatus 1 according to the present embodiment will be described. FIG. 6 is a flowchart showing an example of a procedure of the treatment support process. The procedure in each process to be described below is given merely as an example, and each process may be suitably altered wherever possible. In the procedure to be described below, one or more steps may be suitably omitted, replaced, and/or added according to the embodiment. Since the processing from step S201 to step S203 in FIG. 6 is similar to that from step S101 to step S103 in FIG. 3, a description thereof will be omitted.

### Treatment Support Process

### Step S204

With the warning function 544, the processing circuitry 54 determines whether or not the rest level calculated at step S203 is smaller than a threshold value A.

### Step S205

If the rest level is smaller than the threshold value A (step S204-Yes), the processing circuitry 54 determines that the rest level of the patient P has decreased, and outputs a warning. For example, the processing circuitry 54 displays a warning message on the display 18, and alters the color of a portion in which the rest level is smaller than a threshold value in the graph showing a change in the rest level over time displayed on the display section 181.

On the other hand, if the rest level is equal to or greater than the threshold value A (step S204-No), the processing circuitry 54 determines that the rest level of the patient P has not decreased, and continues monitoring the rest level of the patient P by repeatedly executing the processing form step S201 to step S203 until setting up of the fastener 60 is completed.

Hereinafter, effects of the radiotherapy apparatus 1 including the output apparatus according to the present embodiment will be described.

The radiotherapy apparatus 1 including the output apparatus according to the present embodiment is capable of outputting a warning if the rest level of the patient has changed and decreased below a threshold value. With the above configuration of the radiotherapy apparatus 1 according to the present embodiment, it is possible for the operator to grasp, even if a complaint has not been made by the patient who is feeling pain due to his or her skin being compressed or rubbed by the fastener 60, that the patient is feeling discomfort from the warning, and thereby to take quick and appropriate measures, similarly to the first embodiment.

### Third Embodiment

A third embodiment will be described. In the present embodiment, the configurations of the first embodiment are modified in the following manner. A description of configurations, operations, and effects similar to those of the first embodiment will be omitted. In the present embodiment, a fastener 60 attached to a patient P lying on the bed 30 is specified using a photographing apparatus capable of photographing the patient P, and a fastener 60 that is the cause of a change in the rest level is specified using a result of the specification.

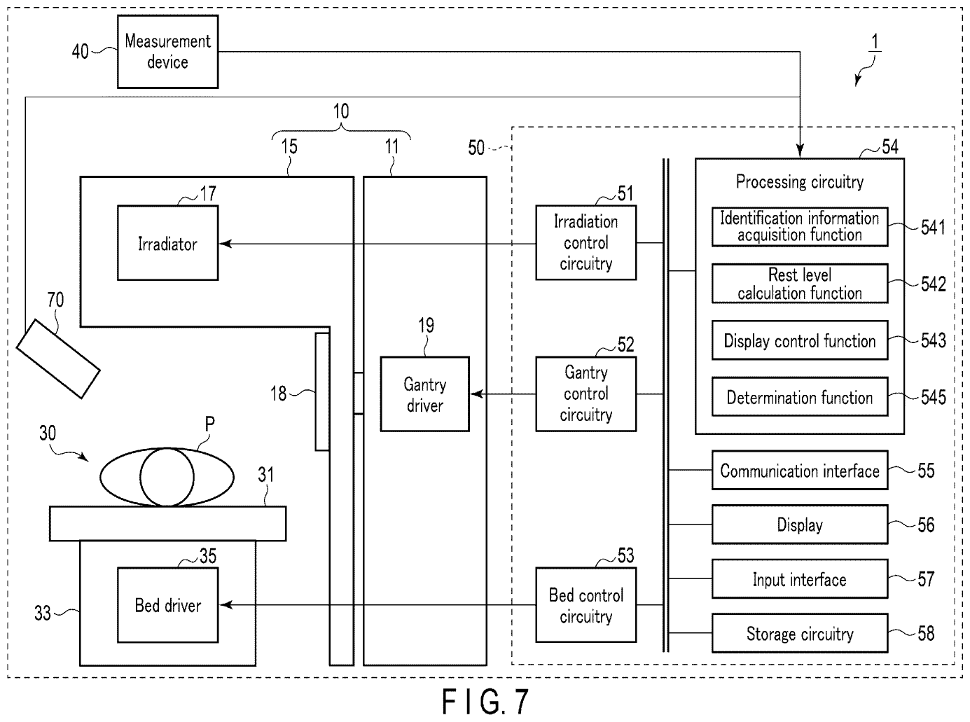

FIG. 7 is a diagram showing a configuration of a radiotherapy apparatus 1 according to the present embodiment. As shown in FIG. 7, in the present embodiment, the radiotherapy apparatus 1 further includes a photographing apparatus 70. The photographing apparatus 70 is connected to the processing circuitry 54 of the console 50 in a wired or wireless manner. The photographing apparatus 70 photographs the state of the patient P placed on the bed 30. The photographing apparatus 70 is, for example, a general camera. The photographing apparatus 70 is fixed at a position in a treatment room where the bed 30 can be photographed from above. The photographing apparatus 70 is fixed to, for example, a ceiling or a wall surface of the treatment room. The photographing apparatus 70 periodically photographs the state of the bed 30 from above under control of the processing circuitry 54, and transmits image data (hereinafter referred to as a “photographed image”) generated by the photographing to the console 50. The photographed image is stored in the storage circuitry 58.

The processing circuitry 54 further executes a determination function 545, in addition to the identification information acquisition function 541, the rest level calculation function 542, and the display control function 543. The processing circuitry 54, which implements the determination function 545, is an example of a determination unit.

With the determination function 545, the processing circuitry 54 acquires a photographed image from the photographing apparatus 70, specifies a fastener 60 attached to the patient using the photographed image, and determines a cause of the change in the rest level based on a result of calculation of the rest level and a result of the specification of the fastener 60. For the specification of the fastener 60, a known image analysis technique, for example, can be used. With the determination function 545, the processing circuitry 54 specifies a timing when each of the fasteners 60 was attached to the patient using the specification result of the fastener 60, and determines, if the rest level changes at a timing when one of the fasteners 60 was attached, that the fastener 60 is the cause of the change in the rest level, and notifies the operator of a determination result.

With the display control function 543, the processing circuitry 54 outputs the determination result of the cause of the change in the rest level determined by the determination function 545 to the display 18, and causes the display 18 to display the determination result.

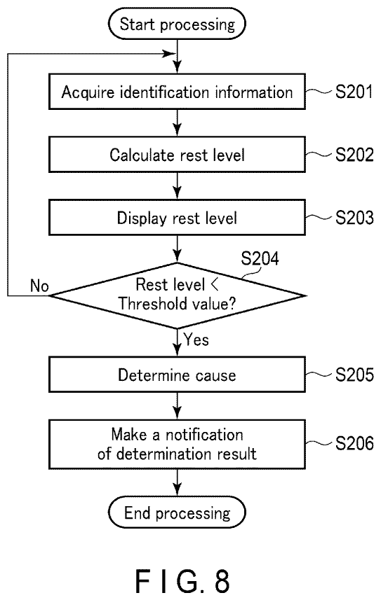

Next, an operation of a treatment support process executed by the radiotherapy apparatus 1 according to the present embodiment will be described. FIG. 8 is a flowchart showing an example of a procedure of the treatment support process. The procedure in each process to be described below is given merely as an example, and each process may be suitably altered wherever possible. In the procedure to be described below, one or more steps may be suitably omitted, replaced, and/or added according to the embodiment. Since the processing from step S301 to step S303 in FIG. 8 is similar to that from step S201 to step S203 in FIG. 6, a description thereof will be omitted.

### Treatment Support Process

### Step S304

With the determination function 545, the processing circuitry 54 determines whether or not the rest level calculated at step S203 is smaller than a predetermined threshold value A. The threshold value is, for example, stored in advance in the storage circuitry 58. If the rest level is equal to or greater than the threshold value A (step S304-No), the processing circuitry 54 determines that the rest level of the patient P has not decreased, and continues monitoring the rest level of the patient P by repeatedly executing the processing from step S301 to step S303 until setting up of the fastener 60 is completed.

### Step S305

If the rest level is smaller than the threshold value A (step S304-Yes), the processing circuitry 54 determines that the rest level of the patient P has decreased. In this case, the processing circuitry 54 acquires recent photographed images generated by the photographing apparatus 70. The photographed images are stored in, for example, the storage circuitry 58.

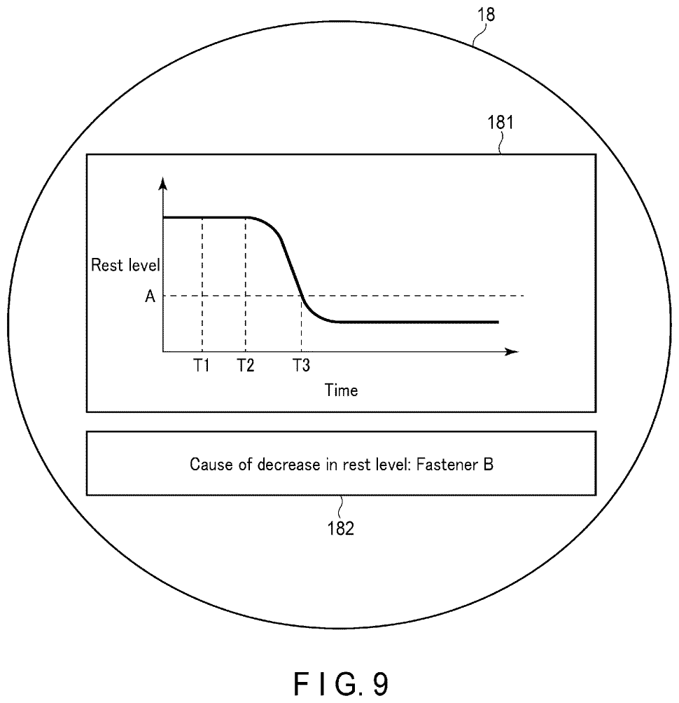

FIG. 9 is an example of a display screen displayed on the display 18. The processing circuitry 54 acquires photographed images obtained in the past few seconds from a point in time when the rest level has decreased below the threshold value A, based on the rest level becoming smaller than the threshold value at time T3. The processing circuitry 54 applies a known image analysis technique to the acquired photographed images, and extracts fasteners 60 included in the photographed images. Thereafter, the extracted fasteners 60 are compared with the shape data, etc. of the fasteners 60 registered in advance to specify the types of the extracted fasteners 60, and the timings when the fasteners 60 were attached is specified. If, for example, a fastener 60 not included in the previous photographed image appears in the subsequent photographed image, the processing circuitry 54 determines that the fastener 60 was attached at that timing. It is assumed herein that a fastener 60A was attached at time T1 and a fastener 60B was attached at time T2. As shown in FIG. 2, for example, the fastener 60A is a fastener for fastening the abdomen of the patient P, and the fastener 60B is a fastener installed below the leg of the patient P for fastening the leg of the patient P.

Subsequently, the processing circuitry 54 determines, based on a result of specification of the timing of attaching the fastener 60 and the timing when the rest level has changed, a cause of the change in the rest level. In the example of FIG. 9, since the rest level does not decrease during the period from time T1 to time T2, the processing circuitry 54 determines that the fastener 60A is not the cause of the decrease in the rest level. On the other hand, since the rest level decreases during the period from time T2 to time T3, the processing circuitry 54 determines that the fastener 60B is not the cause of the decrease in the rest level.

### Step S306

With the display control function 543, the processing circuitry 54 causes the display 18 to display, on a display screen, results of the determination of the cause of the decrease in the rest level by the processing at step S305. As shown in FIG. 9, a cause display section 182 is provided on the display screen. A determination result of the fastener 60 that is the cause of the decrease in the rest level is displayed on the cause display section 182. Herein, the fastener 60B is displayed as the cause of the decrease in the rest level. The operator can easily grasp the fastener 60 that is the cause of the sense of discomfort by checking the cause display section 182.

Hereinafter, effects of the radiotherapy apparatus 1 including the output apparatus according to the present embodiment will be described.

The radiotherapy apparatus 1 including the output apparatus according to the present embodiment is capable of specifying a fastener 60 attached to a patient based on image data obtained by photographing the patient, determining a cause of a change in the rest level based on a calculation result of the rest level and a specification result of the fastener 60, and outputting a determination result of the cause.

With the above-described configuration of the radiotherapy apparatus 1 including the output apparatus according to the present embodiment, it is possible, in the case of using a plurality of fasteners 60, to determine a fastener 60 that is the cause of the sense of discomfort being felt by the patient by specifying a timing when each fastener 60 was attached and specifying a fastener 60 attached at a timing when a rest level has decreased, and to notify the operator of the specified fastener 60. Upon receiving the notification, the operator can correctly attach the specified fastener 60, and it is thereby possible to inhibit a treatment from being started on a patient feeling discomfort.

According to at least one embodiment described above, it is possible to inhibit a treatment from being executed on a patient feeling discomfort.

The radiotherapy apparatus 1 has been described as executing a plurality of functions with a single console; however, a plurality of functions may be executed by separate devices. For example, the functions of the processing circuitry 54 may be installed on different devices in a distributed manner.

The functions have been described as being implemented by single processing circuitry 54; however, the processing circuitry may be configured of a plurality of independent processors in combination, and the functions may be implemented by the processors executing the respective programs. In addition, the functions implemented by the processing circuitry 54 may be implemented as individual hardware circuits.

The term “processor” used in the above explanation refers to, for example, circuitry such as a central processing unit (CPU), a graphics processing unit (GPU), an application-specific integrated circuit (ASIC), a programmable logic device (e.g., a simple programmable logic device (SPLD), a complex programmable logic device (CPLD), a field programmable gate array (FPGA)), etc. If, for example, the processor is a CPU, the processor reads and executes programs stored in storage circuitry to execute the respective functions. On the other hand, if the processor is an ASIC, for example, the functions are directly incorporated into the circuitry of the processor as logic circuits, instead of the programs being stored in the storage circuitry. Each processor of the above embodiments is not necessarily configured as a single circuit, and may be configured by a combination of a plurality of independent circuits to implement its functions. Furthermore, a plurality of components may be integrated into a single processor to implement their functions.

Various embodiments have been described above; however, these embodiments are presented merely as examples, and are not intended to limit the scope of the invention. The embodiments described herein may be practiced in a variety of other forms; furthermore, various omissions, substitutions, and alterations as well as combinations of the embodiments may be made without departing from the spirit of the invention. The accompanying claims and their equivalents are intended to cover such forms or modifications as would fall within the scope and spirit of the inventions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An output apparatus comprising processing circuitry configured to:
acquire identification information for identifying a state of a patient fastened to a radiotherapy apparatus with a fastener;
calculate a rest level of the patient based on the identification information; and
output a change in the rest level over time.

2. The output apparatus according to claim 1, further comprising:
a display configured to display the change in the rest level over time, wherein
the processing circuitry outputs the rest level to the display.

3. The output apparatus according to claim 1, wherein
the processing circuitry is configured to:
acquire, as a reference value, a rest level in a normal state in which the fastener is not attached, and calculate an amount of change in the rest level relative to the reference value; and
output the calculated amount of change in the rest level.

4. The output apparatus according to claim 3, wherein
the normal state is a state during a treatment plan, a state during preparing of the fastener, a waiting state, or a state immediately before the fastener is attached.

5. The output apparatus according to claim 1, wherein
the processing circuitry outputs a warning if the rest level has changed.

6. The output apparatus according to claim 1, wherein
the processing circuitry is configured to:
specify a fastener attached to the patient based on image data obtained by photographing the patient, and determine a cause of the change in the rest level based on a result of the calculating of the rest level and a result of the specifying of the fastener; and
output a result of the determining of the cause of the change in the rest level.

7. A radiotherapy apparatus, comprising:
the output apparatus according to claim 1;
a bed on which the patient is placed;
an irradiator unit configured to emit radiation;
a gantry configured to support the irradiator; and
a measurement device configured to measure the identification information of the patient.
